(19) United States
(12) Patent Application Publication
(10) Pub. No.: US 2026/0083969 A1
(43) Pub. Date: Mar. 26, 2026

(54) ELECTRONIC VISION SYSTEM USING OCULAR DEVICE AND NEURAL DEVICE

(71) Applicant: GLAIVE MEDICAL OPTICS, INC., Carlisle, MA (US)

(72) Inventors: Wayne H. WOODS, JR., Carlisle, MA (US); Alexander J. MELVILLE, Chelmsford, MA (US); Christopher SHELBY, Shreveport, LA (US)

(21) Appl. No.: 19/341,798

(22) Filed: Sep. 26, 2025

Related U.S. Application Data
(60) Provisional application No. 63/699,295, filed on Sep. 26, 2024.
(60) Provisional application No. 63/699,256, filed on Sep. 26, 2024.
(60) Provisional application No. 63/699,293, filed on Sep. 26, 2024.

(51) Int. Cl. A61N 1/36; A61F 9/08

(52) U.S. Cl. CPC A61N 1/3605; A61F 9/08

(57) ABSTRACT

An artificial vision system includes: an ocular device configured to receive incoming light at an eye of a user and wirelessly transmit data based on the incoming light; and a neural device that is configured to receive the data and stimulate a visual cortex of a brain of the user based on the data. Stimulating the visual cortex includes selectively applying electrical pulses to one or more particular parts of the visual cortex to create artificial vision corresponding to the incoming light.

### RELATED APPLICATIONS

The application claims priority to US provisional patent application number 63/699,256, filed September 26, 2024, US provisional patent application number 63/699,293, filed September 26, 2024, and US provisional patent application number 63/699,295, filed September 26, 2024, the contents of each of which are incorporated by reference herein in their entirety.

### BACKGROUND

The present disclosure relates to electronic vision systems. Such systems have a variety of application such as therapeutic (e.g., to treat vision disorders such as blindness), professional, and recreation.

### SUMMARY

In accordance with aspects of the present disclosure, an electronic vision system includes an ocular device and a neural device that work together to provide visual information directly to the brain, bypassing the retina and optical nerve. According to aspects of the present disclosure, the ocular device collects visual data via an imaging system and transmits signals corresponding to the visual data to the neural device which delivers electrical pulses to the brain in a manner that causes the brain to create visual perceptions in the user corresponding to the visual data. In this manner, blindness from a multitude of disparate sources can be treated, including eye injuries, retinal diseases, and age-related degeneration.

In an aspect of the present disclosure, there is an artificial vision system comprising: an ocular device configured to receive incoming light at an eye of a user and wirelessly transmit data based on the incoming light; and a neural device that is configured to receive the data and stimulate a visual cortex of a brain of the user based on the data.

In various embodiments of the artificial vision system, the stimulating the visual cortex comprises selectively applying electrical pulses to one or more particular parts of the visual cortex to create artificial vision corresponding to the incoming light.

In various embodiments of the artificial vision system, the artificial vision system is configured to bypass a retina and optic nerve of the eye.

In various embodiments of the artificial vision system, the ocular device is configured to be implanted in the eye.

In various embodiments of the artificial vision system, the ocular device is configured to be implanted in a capsular bag of the eye, a ciliary sulcus of the eye, or an anterior chamber of the eye.

In various embodiments of the artificial vision system, the ocular device comprises a central body and haptics extending from the central body.

In various embodiments of the artificial vision system, the ocular device comprises an imaging system, control circuitry, a power source, a wireless communication system, and a wireless charging system.

In various embodiments of the artificial vision system, the imaging system is configured to receive the incoming light at the eye and output a signal based on the incoming light, the control circuitry is configured to convert the signal to the data, and the wireless communication system is configured to transmit the data to the neural device.

In various embodiments of the artificial vision system, the wireless communication system is configured to transmit the data directly to the neural device.

In various embodiments of the artificial vision system, the wireless communication system is configured to transmit the data indirectly to the neural device via an external device.

In various embodiments of the artificial vision system, the artificial vision system comprises the external device and the external device is configured to receive the data wirelessly from the ocular device and transmit that data, wired or wirelessly, to the neural device.

In various embodiments of the artificial vision system, the ocular device is devoid of a light projection system.

In various embodiments of the artificial vision system, the ocular device comprises a first ocular device configured to be implanted in a first eye of the user, the artificial vision system further comprises a second ocular device configured to be implanted in a second eye of the user, and the neural device is configured to receive first data from the first ocular device and second data from the second ocular device and apply the electrical pulses to the visual cortex of the brain of the user based on the first data and the second data.

In various embodiments of the artificial vision system, the ocular device comprises a contact lens that is configured to be placed on an exterior surface of the eye.

In various embodiments of the artificial vision system, the ocular device comprises an implantable contact lens that is configured to be implanted in the eye.

In accordance with aspects of the present disclosure, a method comprises implanting the ocular device of the artificial vision system into the eye of the user. The method may further comprise implanting the neural device in a head of the user.

In accordance with aspects of the present disclosure, a method comprises using the artificial vision system to create artificial vision for the user. The method may comprise: generating a signal based on the receiving the incoming light at an imaging system of the ocular device; converting the signal to data; transmitting the data to the neural device; and stimulating the visual cortex of the user using the neural device and based on the data.

### BRIEF DESCRIPTION OF THE DRAWINGS

Various features and advantages of the technology of the present disclosure will be apparent from the following description of particular examples of those technologies, and as illustrated in the accompanying drawings. The drawings are not necessarily to scale; the emphasis instead is placed on illustrating the principles of the technological concepts. In the drawings, like reference characters may refer to the same parts throughout the different views. The drawings depict only illustrative examples of the present disclosure, and are not limiting in scope.

FIG. 1 shows an electronic vision system in accordance with aspects of the present disclosure.

FIG. 2 shows diagrammatic cross sectional views of components of the electronic vision system of FIG. 1 in accordance with aspects of the present disclosure.

FIG. 3 shows an example of an ocular device implanted in an eye in accordance with aspects of the present disclosure.

FIG. 4 shows an example implementation of an electronic vision system in accordance with aspects of the present disclosure.

FIG. 5 shows another example implementation of an electronic vision system in accordance with aspects of the present disclosure.

FIG. 6 illustrates wireless charging in accordance with aspects of the present disclosure.

FIG. 7 shows a flowchart of an exemplary method in accordance with aspects of the present disclosure.

### DETAILED DESCRIPTION

The particulars shown herein are by way of example and for purposes of illustrative discussion of the embodiments of the present disclosure only and are presented in the cause of providing what is believed to be the most useful and readily understood description of the principles and conceptual aspects of the present disclosure. In this regard, no attempt is made to show structural details of the present disclosure in more detail than is necessary for the fundamental understanding of the present disclosure, the description taken with the drawings making apparent to those skilled in the art how the several forms of the present disclosure may be embodied in practice.

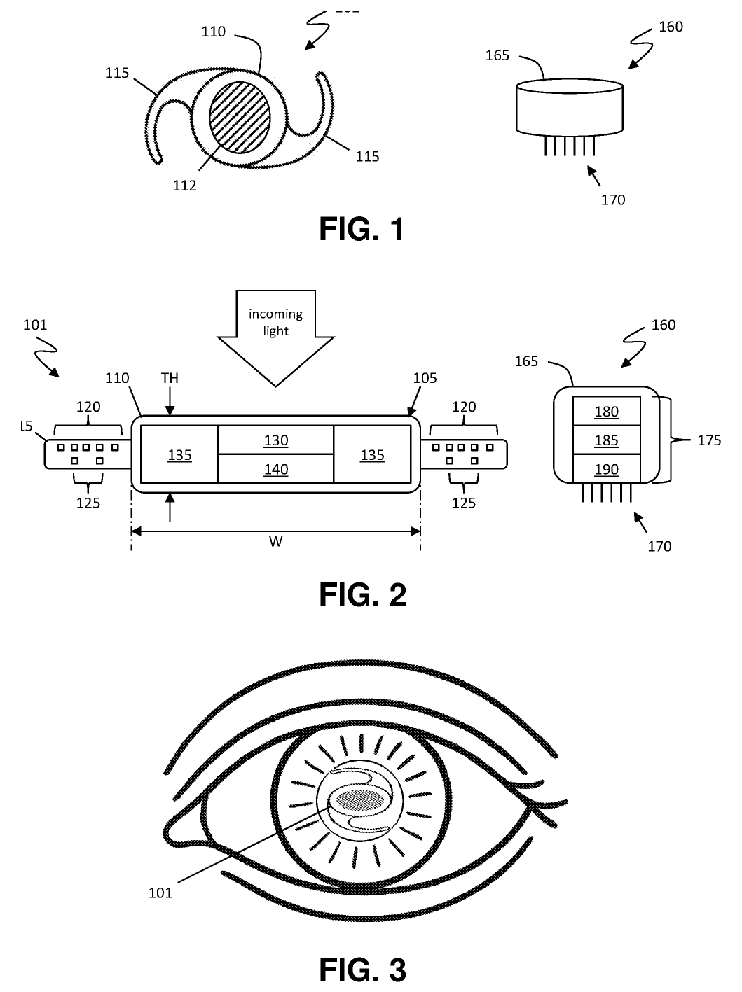

FIG. 1 shows an example of an electronic vision system in accordance with aspects of the present disclosure. In embodiments, the electronic vision system includes an ocular device 101 and a neural device 160 that work together to provide visual information directly to the brain, bypassing the retina and optical nerve. In embodiments, the ocular device 101 is in the form of an electronic device that is configured to be implanted in the eye of a user and comprises a body 105 that has a central portion 110 and haptics 115 with electronics 112 in the body 105. In embodiments, the neural device 160 is in the form of a neural implant comprising a body 165 and one or more electrodes 170 that is configured to be implanted in the user for operative connection to the brain of the user. The ocular device 101 and the neural device 160 are depicted diagrammatically in FIG. 1 and are not limited to the shapes shown in FIG. 1. For example, the central portion 110 and/or the haptics 115 of the ocular device 101 may be shaped differently than shown in FIG. 1. Similarly, the body 165 and/or the electrodes 170 may be shaped differently than shown in FIG. 1.

FIG. 2 shows diagrammatic cross sectional views of the ocular device 101 and the neural device 160 of FIG. 1. In embodiments, and as shown in FIGS. 1 and 2, the body 105 comprises haptics 115 in the form of wings or tabs that each extend outward from the central portion 110. The body 105 may be made in the form of a single piece composed of materials such as acrylic and/or silicone material that encapsulates the electronics 112. In another example, the body 105 may be made in the form of a multi-piece (e.g., two piece) case that can be opened and closed and that houses the electronics of the ocular device 101.

In embodiments, the electronics 112 include a wireless charging coil 120, a wireless communication antenna 125, an imaging system 130, a power source 135, and control circuitry 140. In some configurations, the charging coil 120 and the wireless communication antenna 125 are embedded in one or both of the haptics 115 and the imaging system 130, power source 135, and control circuitry 140 are integrated in chip stack contained in the central portion 110 of the body 105, although other arrangements may be utilized. In some embodiments, the functions of the charging coil 220 and the wireless communication antenna 225 are combined in a single coil of electrical conductor (e.g., wire). In some embodiments, the charging coil 220 and the wireless communication antenna 225 are formed in the shape of a coil wrapped around an interior of the body 205.

In accordance with aspects of the present disclosure, the imaging system 130 is configured to receive incoming light from outside the eye when the ocular device 101 is implanted in an eye of a user, e.g., as shown in FIG. 3. In embodiments, the imaging system 130 receives incoming light from outside the eye and provides input to the control circuitry 140 based on the received light. The imaging system 130 may comprise any suitable type of on-chip imaging technology, such as a charge-coupled device (CCD). The imaging system 130 may also include specialized local lens structures to enhance functionality of the imaging chip. In some embodiments, the output of the imaging system 130 is a time-dependent electronic signal to the control circuitry 140.

In accordance with aspects of the present disclosure, the control circuitry 140 is configured to receive signals that are output by the imaging system 130 (e.g., the signals corresponding to the incoming light received by the imaging system 130), to convert these signals to visual data, and to transmit the visual data to the neural device 160. In embodiments, the control circuitry 140 uses the wireless communication antenna 125 to wirelessly transmit the visual data to the neural device 160. The wireless communication antenna 125 may be in the haptics 115 as shown in FIG. 2 or alternatively may be in a chip or chip stack that contains the control circuitry 140, or both.

The control circuitry 140 may comprise a controller, a central processing unit (CPU), an application-specific integrated circuit (ASIC), a field-programmable gate array (FPGA), a system-on-chip (SoC), or other processing device or computing device for processing executable instructions. The electronics 112 may be composed of sub-circuits which may be on disparate chip materials and made with disparate technologies, such as Si, SiC, SiN, InP, GaAs, etc. This integrated system can be stacked in as shown in FIG. 2, with the connections between circuit elements being formed using BGA/C4/micro-BGA, through substrate (or silicon) vias (TSVs), and micro-TSVs. Physical connections between layers can be through solder or oxide bonding techniques. In one example, the control circuitry 140 comprises a printed circuit board and the imaging system 130 may comprise one or more chips that are physically and electronically connected to the printed circuit board.

In various embodiments, the ocular device 101 is specifically configured via its shape and size to be implanted in a capsular bag of an eye, a ciliary sulcus of an eye, or an anterior chamber of an eye, e.g., as shown and described in US Patent Application Publications No. 2024/0138673 and 2025/0099299, the contents of each of which are incorporated by reference herein in their entirety. In this regard, in the ocular device 101 sub-circuit chips of the electronics 112 may be thinned using wafer thinning techniques to be thin enough such that the entire system is such that the thickness dimension TH satisfies the expression 1 mm <= TH <=3 mm. These techniques are employed in stacked memory chips with wafers thinned to less than 20 μm thick and bonded to other wafers and connecting micro-TSVs are made between active layers that are 10 μm to 20 μm tall. The ocular device 101 may be constructed such that the width dimension W satisfies the expression 1 mm <= W <=10 mm. An ocular device 101 having these dimensions TH and W is suitable for implanting an eye (e.g., a human eye), such as shown in FIG. 3 and as shown and described in US Patent Application Publications No. 2024/0138673 and 2025/0099299. In embodiments, because the vision system of the present disclosure bypasses the retina and optic nerve, the ocular device 101 does not include a light projection system (e.g., LED, OPA, etc.) for projecting images onto the retina of the user. This enables the ocular device 101 to be smaller (e.g., in both W and TH) and use less power than devices that include an image projection system for projecting images onto the retina of the user. Being smaller provides the advantage of being less invasive in the user’s eye, and using less power provides the advantages of longer batter life, less heat generation, or even further reduced size by shrinking the power source 135.

Because the ocular device 101 in accordance with aspects of the present disclosure is configured to be implanted inside the eye, the ocular device 101 moves with the eye during movement of the eye. This is in contrast to eyeglasses that do not move with the movement of the eye and contact lenses that may slide across the surface of the eye and thus become misaligned with the pupil and also require frequent reinsertion and cleaning, and which can lead to poor air circulation to the surface of the eye. In this manner, when the ocular device 101 is implanted in an eye (e.g., in the manner shown and described in US Patent Application Publications No. 2024/0138673 and 2025/0099299), the imaging system 130 is aimed where the user aims their eye (e.g., when a user moves their eye to look in a direction), which is an improvement over an image sensor that is located on eyeglasses or a contact lens, since such an image sensor that is located on eyeglasses or a contact lens is not always aimed where the user aims their eye or that require additional cleaning, and other complications as described previously.

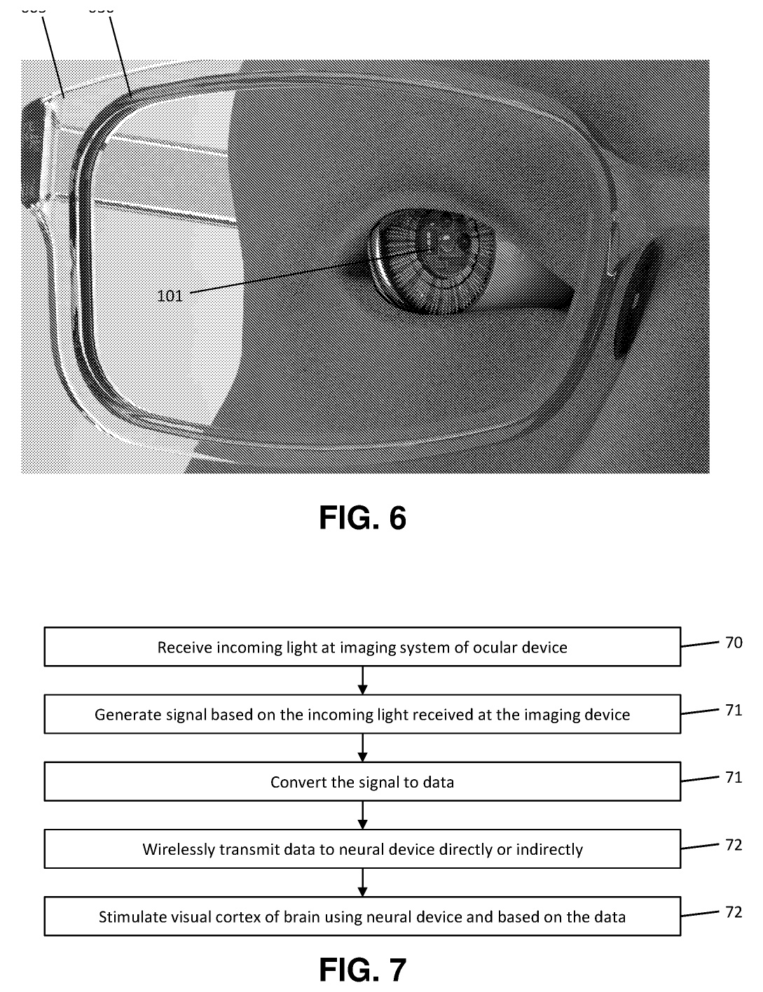

In embodiments, the wireless charging coil 120 comprises one or more inductive coupling coils and the power source 135 comprises a rechargeable battery that can be wirelessly recharged through inductive coupling using the wireless charging coil 120. In this regard, although not shown, the wireless charging coil 120 may be operatively connected to the power source 135, e.g., directly or indirectly via the control circuitry 140. The wireless charging coil 120 may be used to wirelessly charge the power source 135 using an external charging device, e.g., as shown in FIG. 6. The wireless communication antenna 125 may be in the haptics 115, in the central body 110, or in a chip or chip stack that contains the control circuitry 140, or any combination thereof. In some embodiments, the power source 135 does not store an appreciable amount of electrical power or the power source 135 is omitted altogether. In such embodiments, the ocular device 101 may be configured to use the electronics only when being wirelessly powered by an external device such as eyeglasses 605 shown in FIG. 6. In such embodiments, the ocular device 101 may be configured to automatically turn on or turn off the electronics, respectively based on the user donning or doffing the external device that wirelessly provides power to the ocular device 101.

In accordance with aspects of the present disclosure, and with continued reference to FIG. 2, the neural device 160 comprises a body 165 and one or more electrodes 170 and may be specifically configured to be implanted in a user for electrical stimulation of the brain of the user. The body 165 may be composed of any material of combination of materials that is suitable for encasing electronics 175 and being implanted in and/or on a body (e.g., a human body). In various embodiments, the electronics 175 in the neural device 160 comprise a communication system 180, a power system 185, and control circuitry 190. The communication system 180, the power system 185, and the control circuitry 190 may be formed using semiconductor technology and may comprise a chip or a chip-stack, e.g., similar to the description of the electronics 112 of the ocular device 101.

In accordance with aspects of the present disclosure, the communication system 180 is configured to receive data transmitted to the neural device 160 from the ocular device 101. The communication system 180 may comprise one or more wireless antennas formed in a chip (or chip-stack) or embedded in the body 165 separately from the chip (or chip-stack), these one or more wireless antennas being configured to facilitate wireless communication with the wireless communication antenna 125 of the ocular device 101.

In accordance with aspects of the present disclosure, the power system 185 is configured to provide electrical power to the electrical components of the neural device 160. In embodiments, the power system 185 comprises a rechargeable battery that is rechargeable via a wired connection (e.g., via a physical connection to a charging port in the body 165) or wirelessly (e.g., via inductive coupling using a wireless charging coil embedded in the body 165).

In accordance with aspects of the present disclosure, the control circuitry 190 is configured to receive data from the ocular device 101 and, based on the received data, provide electrical pulses to selected ones of the electrodes 170 for the purpose of electrically stimulating portions of the brain to which the electrodes 170 are operatively connected. In embodiments, the neural device 160 is implanted in a user such that the electrodes 170 are operatively connected to the visual cortex of the brain of the user. In this manner, the control circuitry 190 is configured to control the delivery of electrical pulses to neurons in the visual cortex via the electrodes 170. These electrical pulses are generated based on signals generated by the imaging system 130, which are translated into a form the brain can interpret as light, creating a pattern of bright dots called phosphenes. The brain then processes these phosphenes, creating a form of vision for the user.

In accordance with aspects of the present disclosure, the electrodes 170 comprise electrically conductive material such as metal, alloy, etc. The electrodes 170 may comprise microelectrode array that is configured to deliver electrical pulses to specific neurons in the visual cortex of the user in which the neural device 160 is implanted. Stimulating these neurons creates perceived flashes of light known as phosphenes, which are like artificial pixels of vision, and the brain interprets these patterned phosphenes as a visual image.

In accordance with aspects of the present disclosure, the electrical pulses that are applied to the visual cortex via the electrodes 170 in the neural device 160 are generated based on the incoming light received by the imaging system 130 in the ocular implant 101. In various embodiments, the control circuitry 140 of the ocular device 101 converts the signals from the imaging system 130 to data and wirelessly transmits the data to the neural device 160. In one example, the output of the imaging system 130 is a time-dependent electronic signal that the control circuitry 140 translates to visual data. The ocular device 101 wirelessly transmits this visual data to the neural device 160. In response to receiving this visual data, the control circuitry 190 of the neural device 160 selectively applies electrical power to respective ones of the electrodes 170, which applies electrical pulses to the visual cortex of the user based on the visual data, which creates a pattern of phosphenes that corresponds to the incoming light received by the imaging system 130 in the ocular implant 101. The brain then processes these phosphenes, creating a form of vision for the user.

In some embodiments, the control circuitry 190 of the neural device 160 translates the signals from the imaging system 130 to the visual data. In these embodiments, the processing associated with translating signals from the imaging system 130 to visual data is offloaded from the ocular device 101 to the neural device 160, which enables minimizing the size of the control circuitry 140 of the ocular device 101, which in turn enables minimizing the size and power consumption of the ocular device 101. In these embodiments, the control circuitry 140 of the ocular device 101 receives the signals from the imaging system 130 and controls transmitting this data to the neural device 160, the control circuitry 190 of which translates this data to the visual data that is used to create electrical pulses for stimulating the visual cortex.

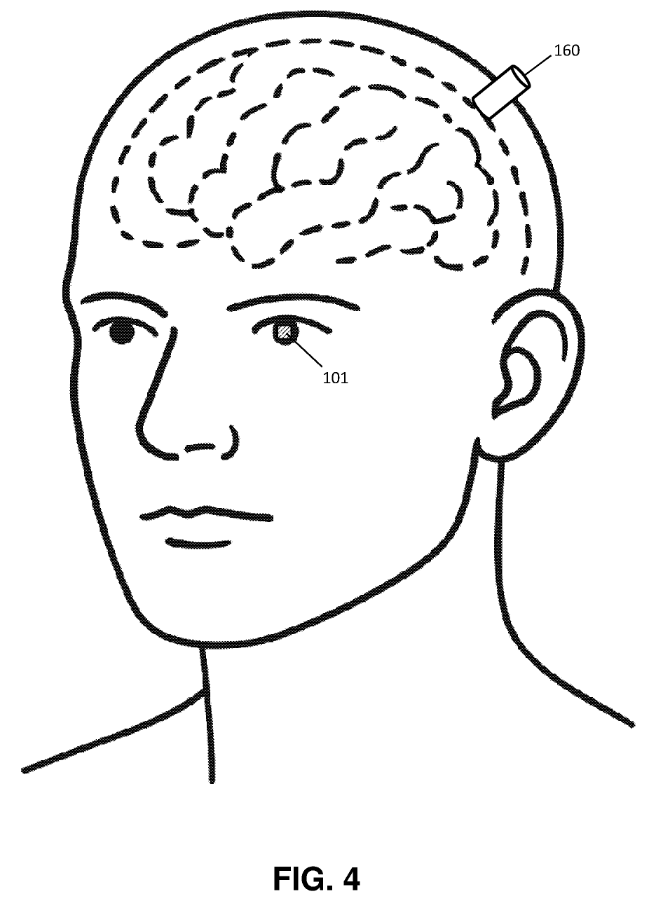

FIG. 4 shows an example implementation of an electronic vision system in accordance with aspects of the present disclosure. In this example, the ocular device 101 is implanted in an eye of a user and the neural device 160 is implanted in the user’s head such that the electrodes 170 (not shown in FIG. 4) contact the visual cortex of the brain. In this example, the ocular device 101 communicates directly with the neural device 160 using wireless communication between the two.

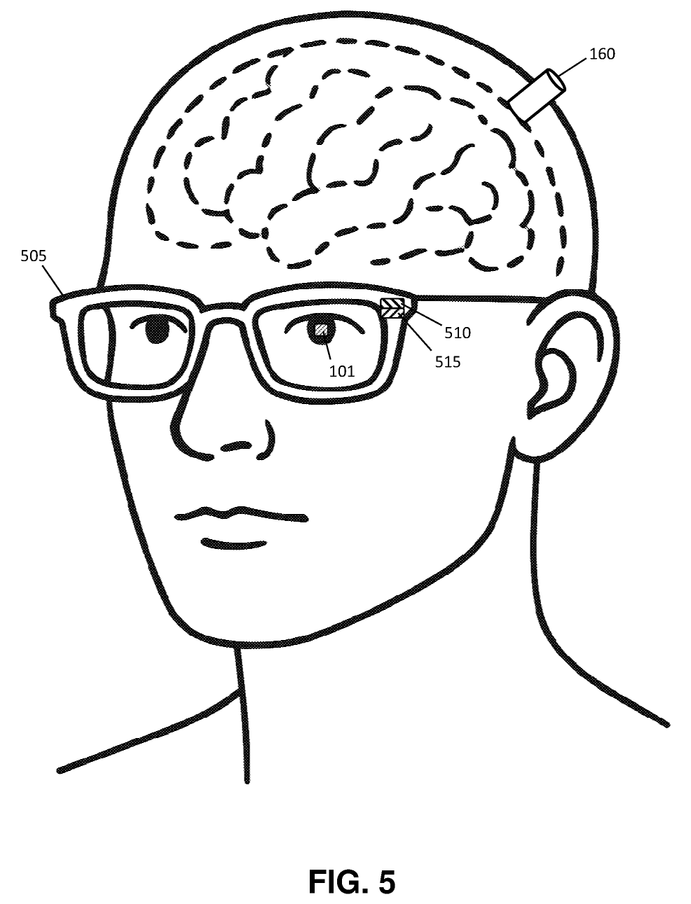

FIG. 5 shows another example implementation of an electronic vision system in accordance with aspects of the present disclosure. In this example, the ocular device 101 is implanted in an eye of a user and the neural device 160 is implanted in the user’s head such that the electrodes 170 (not shown in FIG. 4) contact the visual cortex of the brain. In this example, the ocular device 101 communicates indirectly with the neural device 160 via an external device 505 worn by the user. The external device 505 may comprise eyeglasses, an eyepatch, a headband, or similar device that is worn on the head of the user.

In embodiments, the external device 505 include a communication system 510. In one example, the communication system 510 comprises one or more wireless antennas configured to wirelessly receive data from the ocular device 101 and wirelessly transmit the data to the neural device 160. In another example, the communication system 510 comprises one or more wireless antennas configured to wirelessly receive data from the ocular device 101 and further comprises a wired connection to the neural device 160 for transmitting the data to the neural device 160. In both examples, the communication system 510 acts as a data transmission relay between the ocular device 101 and the neural device 160, which permits minimizing the size of the power and antenna components of the ocular device 101.

With continued reference to FIG. 5, in some embodiments the external device 505 include control circuitry 515 that may be configured to perform one or more functions described herein with respect to the control circuitry 140 of the ocular device 101 and/or the control circuitry 190 of the neural device 160. In one example, the processing associated with translating signals from the imaging system 130 to visual data is offloaded from the ocular device 101 and/or neural device 160 to the control circuitry 515 on the external device 505, which enables minimizing the size of the control circuitry 140 of the ocular device 101 and/or the control circuitry 190 of the neural device 160, which in turn enables minimizing the size and power consumption of the ocular device 101 and/or the neural device 160. In these embodiments, the control circuitry 140 of the ocular device 101 receives the signals from the imaging system 130 and controls transmitting this data to the external device 505. The control circuitry 515 of the external device 505 then translates these signals to the visual data and controls transmitting the visual data to the neural device 160. The control circuitry 190 then creates electrical pulses for stimulating the visual cortex based on the visual data received from the external device 505.

FIG. 6 illustrates wireless charging in accordance with aspects of the present disclosure. In various embodiments, an external device such as eyeglasses 605 includes a wireless charging system 630 that is configured to wirelessly charge the a power source 135 of the ocular device 101. In one example, the wireless charging system 630 comprises a charging coil that cooperates with the charging coil 120 in the ocular device 101 to perform inductive charging of the power source 135 of the ocular device 101. The eyeglasses 605 may include its own power source, such as an on-board battery or wired connection to an external power source, that powers the wireless charging of the power source 135 of the ocular device 101. In embodiments in which the power source 135 does not store an appreciable amount of power or is omitted, the wireless charging system 630 is configured to wirelessly provide power to the ocular device 101 for operating the electronics during the time that the power is wirelessly provided. The external device 505 of FIG. 5 may include the wireless charging system 630 of FIG. 6.

In some embodiments, the ocular device 101 comprises a contact lens that is configured to be placed on the exterior surface of the eye of the user, or an implantable contact lens that is configured to be implanted in the eye. In these embodiments, the contact lens comprises a wireless charging coil 120, wireless communication antenna 125, imaging system 130, power source 135, and control circuitry 140 as described herein, and these components function in a similar manner as described herein. These components may have different sizes, shapes, and locations in a contact lens due to the different form factors of contact lenses.

FIG. 7 shows a flowchart of an exemplary method in accordance with aspects of the present disclosure. Steps of the method (also referred to as operations) may be carried out using the system of FIGS. 1 and 2 and are described with reference to elements depicted in FIG. 2.

Step 705 comprises receiving incoming light at the imaging system 130 of the ocular device 101, which may be implanted in an eye of a user. Step 710 comprises generating one or more signals based on the incoming light received at the imaging device. Step 715 comprises converting the signal to data. Step 720 comprises wirelessly transmitting the data to the neural device 160, e.g., directly from the ocular device 101 to the neural device 160 or indirectly from the ocular device 101 to an external device 505 to the neural device 160. Step 725 comprises stimulating a visual cortex of the user’s brain using neural device 160 and based on the data.

Various embodiments include methods of using an artificial vision system in accordance with aspects of the present disclosure. One such method is described in FIG. 7. Various embodiments include methods of implanting an artificial vision system in accordance with aspects of the present disclosure. Such methods may include implanting the ocular device into the eye of the user and/or implanting the neural device in the head of the user.

Implementations of the present disclosure provide superior optical training for the brain making use of the body’s natural eye movements including small rapid changes (e.g., microsaccades). Implementations of the present disclosure may be used to provide a stereoscopic artificial vision system by using a respective ocular device in each eye of the user. Implementations of the present disclosure that utilize ocular implants enable an artificial vision system that tracks with (e.g., remains aligned with) the movement of the eye since the ocular device is implanted in the eye, as opposed to external hardware moves with the user’s head but not necessarily with the user’s eyes.

Various embodiments may include wired (e.g., fiber optic) or wireless communication between the ocular implant and the neural implant. For wired communication, an electrically conductive and/or fiber optic path may be provided that operative connects the ocular implant and the neural implant for the purpose of passing data between the ocular implant and the neural implant. Various embodiments enable wireless communication between ocular implant and the brain via the neural implant. Various embodiments enable wireless communication between a contact lens implementation of the ocular device and the neural implant. Various embodiments enable wired (e.g. fiber optic or electrical cables) or wireless communication between the ocular device and its associated accessory hardware and the neural implant.

In various embodiments, the ocular device 101 is an electronic device (eDevice) comprising an electronic ocular device or an electronic contact lens (eContact) sharing data with the neural device 160 in the form of a brain implant. In embodiments, the eDevice and brain implant work together to provide visual information directly to the brain, bypassing the retina and optical nerve. In this fashion, blindness from a multitude of disparate sources can be treated, including eye injuries, retinal diseases, and age-related degeneration. In embodiments, the eDevice collects the visual data via a camera or image sensor (e.g., imaging system 130), sends the data to the brain implant, and the brain implant delivers the signals to the brain. This pathway can be accomplished either with or without the use of the accessories that power and communicate with the eDevice. In implementations that do not include an external device (e.g., external device 505), the eDevice communicates directly with the brain implant. This pathway may include an additional communication channel built into the eDevice communication chip. In implementations that include an external device (e.g., external device 505), the eDevice communicates with the external device 505, and the external device 505 communicates with the brain implant. This may utilize an additional communication channel in the eDevice depending on the data rate and the eDevice, for example. These implementations may utilize an additional communication channel in the external device 505, but the larger size and reduced limitations of the external device 505 compared to the eDevice makes this a less complicated solution. In implementations in which respective eDevices are used in respective ones of both eyes of the user, then the provided vision is stereoscopic. As described here, since either eDevice is located on the eye itself and moves with the eye, it is advantageously eye-tracking and does not require resources devoted to digitally tracking the motion of the eye. The brain is trained using the eyes’ natural movements which include deliberate eye direction changes as well as microsaccades or small rapid involuntary eye movements. Our brains use all of these motions naturally in brain training in the visual cortex from the time we are babies. This data from the eyes is the superior data set for visual cortex retraining of vision impaired patients. With the interest in vision to brain electronic solutions, implementations of the present disclosure thus provide the best-case solution for vision-to-brain interface.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. An artificial vision system comprising:
an ocular device configured to receive incoming light at an eye of a user and wirelessly transmit data based on the incoming light; and
a neural device that is configured to receive the data and stimulate a visual cortex of a brain of the user based on the data.

2. The artificial vision system of claim 1, wherein the stimulating the visual cortex comprises selectively applying electrical pulses to one or more particular parts of the visual cortex to create artificial vision corresponding to the incoming light.

3. The artificial vision system of claim 2, wherein the artificial vision system is configured to bypass a retina and optic nerve of the eye.

4. The artificial vision system of claim 1, wherein the ocular device is configured to be implanted in the eye.

5. The artificial vision system of claim 4, wherein the ocular device is configured to be implanted in a capsular bag of the eye, a ciliary sulcus of the eye, or an anterior chamber of the eye.

6. The artificial vision system of claim 4, wherein the ocular device comprises a central body and haptics extending from the central body.

7. The artificial vision system of claim 4, wherein the ocular device comprises an imaging system, control circuitry, a power source, a wireless communication system, and a wireless charging system.

8. The artificial vision system of claim 7, wherein:
the imaging system is configured to receive the incoming light at the eye and output a signal based on the incoming light;
the control circuitry is configured to convert the signal to the data; and
the wireless communication system is configured to transmit the data to the neural device.

9. The artificial vision system of claim 8, wherein the wireless communication system is configured to transmit the data directly to the neural device.

10. The artificial vision system of claim 8, wherein the wireless communication system is configured to transmit the data indirectly to the neural device via an external device.

11. The artificial vision system of claim 10, wherein the artificial vision system comprises the external device and the external device is configured to:
receive the data wirelessly from the ocular device; and
transmits that data, wired or wirelessly, to the neural device.

12. The artificial vision system of claim 7, wherein the ocular device is devoid of a light projection system.

13. The artificial vision system of claim 1, wherein:
the ocular device comprises a first ocular device configured to be implanted in a first eye of the user;
the artificial vision system further comprises a second ocular device configured to be implanted in a second eye of the user; and
the neural device is configured to receive first data from the first ocular device and second data from the second ocular device and apply the electrical pulses to the visual cortex of the brain of the user based on the first data and the second data.

14. The artificial vision system of claim 1, wherein the ocular device comprises a contact lens that is configured to be placed on an exterior surface of the eye.

15. The artificial vision system of claim 1, wherein the ocular device comprises an implantable contact lens that is configured to be implanted in the eye.

16. A method comprising implanting the ocular device of claim 1 into the eye of the user.

17. The method of claim 16, further comprising implanting the neural device in a head of the user.

18. A method of using the artificial vision system of claim 1 to create artificial vision for the user.

19. The method of claim 18, further comprising:
generating a signal based on the receiving the incoming light at an imaging system of the ocular device;
converting the signal to data;
transmitting the data to the neural device; and
stimulating the visual cortex of the user using the neural device and based on the data.
